(19) United States
(12) Patent Application Publication
(10) Pub. No.: US 2020/0363383 A1
(43) Pub. Date: Nov. 19, 2020

(54) SYSTEM AND METHOD FOR CALIBRATING A GAS DETECTING DEVICE

(71) Applicant: Kam Fu Chow, Kowloon Bay (HK)

(72) Inventor: Kam Fu Chow, Kowloon Bay (HK)

(21) Appl. No.: 16/639,892

(22) PCT Filed: Aug. 23, 2018
(86) PCT No.: PCT/CN2018/101857

§ 371 Date: Feb. 18, 2020

(30) Foreign Application Priority Data
Aug. 25, 2017 (HK) 17108569.5

(51) Int. Cl. G01N 33/00; G01N 27/12; G01N 27/416

(52) U.S. Cl. CPC G01N 33/0006; G01N 27/4163; G01N 27/125

(57) ABSTRACT

A system and a method for calibrating a gas detecting device. The method comprises the steps of providing the gas detecting device in an enclosed gas chamber filled with a gas having a predetermined composition; and activating the gas detecting device to operate in a calibration mode.

### TECHNICAL FIELD

The present invention relates to a system and a method for calibrating a gas detecting device, and particularly, although not exclusively, to a system and a method for simultaneously calibrating multiple gas detecting devices.

### BACKGROUND

Air pollution has been a vexing problem ever since combustion became the dominant power source for human technologies. Industrial and chemical operation, coupled to underground exploitation such as mining, also releases poisonous gases which risk the health of the workers and the general public. In order to ensure their safety, it is important to inform the people of the air condition and the intensity of potential pollutant gases in the area.

Electrochemical gas detectors may be used to measure the intensity of different gas substances. An electrical current, which is proportional to gas concentration, is measured upon a reduction or oxidation reaction involving the gas type. A functioning gas detector is important to ensure health safety of those potentially exposed to air pollutant.

### SUMMARY OF THE INVENTION

In accordance with a first aspect of the present invention, there is provided a method of calibrating a gas detecting device, comprising the steps of: providing the gas detecting device in an enclosed gas chamber filled with a gas having a predetermined composition; and activating the gas detecting device to operate in a calibration mode.

In an embodiment of the first aspect, the enclosed gas chamber is filled with an inert gas.

In an embodiment of the first aspect, the method further comprises the step of injecting the gas to fill up the enclosed gas chamber and depleting air in the enclosed gas chamber, wherein a gas pressure within the enclosed gas chamber is higher than that of the environment.

In an embodiment of the first aspect, the method further comprises the step of adjusting the gas pressure within the enclosed gas chamber.

In an embodiment of the first aspect, the step of activating the gas detecting device to the calibration mode further comprising the step of wirelessly controlling the gas detecting device with a wireless controller.

In an embodiment of the first aspect, the step of activating the gas detecting device to the calibration mode further comprising the step of manually manipulating of the gas detecting device enclosed within the gas chamber.

In an embodiment of the first aspect, the method further comprises the step of providing at least one additional gas detecting device in the enclosed gas chamber such that the gas detecting device and the at least one additional gas detecting device are simultaneously calibrated.

In an embodiment of the first aspect, the method further comprises the step of transporting a mobile calibration system including the gas chamber and a gas cylinder which contains the gas.

In an embodiment of the first aspect, the gas chamber and the gas cylinder is carried on a mobile mechanical structure.

In accordance with a second aspect of the present invention, there is provided a method of identifying a malfunctioning gas detecting device, comprising the step of: calibrating a gas detecting device using a method in accordance with the first aspect; replacing the inert gas in the gas chamber with standard gas including standard gas composition; determining whether the gas detecting device is malfunctioning based on a comparison of a detection result associated with the standard gas composition and a predetermined record.

In an embodiment of the second aspect, the predetermined record includes a factory guaranteed tolerance.

In an embodiment of the second aspect, the method further comprises the step of waiting for a predetermined period of time prior to recording the detection result.

In an embodiment of the second aspect, the step of replacing the inert gas in the gas chamber with standard gas comprising the step of flushing the gas chamber with the standard gas for multiple times.

In accordance with a third aspect of the present invention, there is provided a system for calibrating a gas detecting device, comprising an enclosed gas chamber arranged to accommodate the gas detecting device therein, wherein the enclosed gas chamber is arranged to be filled with a gas having a predetermined composition; and wherein the gas detecting device is arranged to operate in a calibration mode.

In an embodiment of the third aspect, the enclosed gas chamber is filled with an inert gas.

In an embodiment of the third aspect, the system further comprises a gas supply arranged to inject the gas to fill up the enclosed gas chamber and to deplete air in the enclosed gas chamber, wherein a gas pressure within the enclosed gas chamber is higher than that of the environment.

In an embodiment of the third aspect, the gas supply includes a gas cylinder.

In an embodiment of the third aspect, the system further comprises at least one valve and/or regulator arrange to control a fluid communication between the gas chamber and the gas supply.

In an embodiment of the third aspect, the system further comprises a wireless controller arranged to wirelessly control the gas detecting device.

In an embodiment of the third aspect, the system further comprises a flexible structure provided on a wall of the gas chamber arranged to facilitate a manual manipulation of the gas detecting device enclosed within the gas chamber by a user.

In an embodiment of the third aspect, the flexible structure includes a glove.

In an embodiment of the third aspect, the enclosed gas chamber is further arranged to accommodate at least one additional gas detecting device, wherein the gas detecting device and the at least one additional gas detecting device are simultaneously calibrated.

In an embodiment of the third aspect, the system further comprises a mobile mechanical structure arranged to accommodate the gas chamber, the wireless controller and the gas supply.

In an embodiment of the third aspect, the system further comprises a standard gas supply arranged to facilitate a determination of a malfunctioning gas detecting device.

In an embodiment of the third aspect, the gas detecting device comprises a metal oxide semiconductor sensor and/or a electrochemical sensor.

In accordance with a fourth aspect of the present invention, there is provided a system for calibrating a plurality of gas detecting devices, comprising: a gas chamber arranged to accommodate the plurality of gas detecting devices, wherein the gas chamber includes a plurality of valves arranged to facilitate a fluid communication between an internal cavity of the gas chamber and an external environment or a supply of an inert gas, and a flexible structure provided on a wall of the gas chamber for facilitating a manual manipulation of the plurality of gas detecting devices enclosed within the gas chamber by a user; a wireless controller arranged to wirelessly control the plurality of gas detecting devices disposed within the gas chamber; and a mobile mechanical structure arrange to accommodate the gas chamber, the wireless controller and a container arranged to supply the inert gas to the gas chamber; wherein the gas chamber is arranged to define an enclosed gas chamber filled with the inert gas having a predetermined composition so as to facilitate a calibration process performed by each of the plurality of gas detecting devices operating in a calibration mode in response to the control by the wireless controller or the manual manipulation by the user.

The calibration system in accordance with the abovementioned aspects is advantageous in that the system may be implemented onto a trolley for higher mobility and easier storage, and offers a convenient on-site calibration method.

In addition, multiple gas detection devices may be calibrated in a single (or fewer) batch process which may effectively reduce the amount of gas being consumed for the calibration process. Advantageously, this may also reduce the time and cost for calibrating a large number of gas detecting devices.

### BRIEF DESCRIPTION OF THE DRAWINGS

Embodiments of the present invention will now be described, by way of example, with reference to the accompanying drawings in which:

FIG. 1a is a front view of a system for calibrating a gas detecting device in accordance with one embodiment of the present invention;

FIG. 1b is a top view of the system of FIG. 1a;

FIG. 1c is a side view of the system of FIG. 1a;

FIG. 2 is a perspective view of a gas chamber of the system of FIG. 1a;

FIG. 3 is a front view of a system for calibrating a gas detecting device in accordance with an alternate embodiment of the present invention;

FIG. 4 is a front view of the system of FIG. 1a, wherein multiple gas detecting devices are placed within the gas chamber undergoing a calibration process; and

FIG. 5 is a flow chart illustrating a method of calibrating the gas detecting devices, and a method to identify defective gas detecting device using the system in FIG. 1a in accordance with an embodiment of the present invention.

### DETAILED DESCRIPTION OF THE PREFERRED EMBODIMENT

There are several reasons why gas detecting devices must be calibrated from time to time. The major reason being is that gas detectors are often operated in harsh environment (e.g. extreme temperatures, humidities etc.). These extreme conditions plus aging of devices would often yield incorrect reading of gas concentration. In situation where it involves multiple gas detecting devices to be calibrated, it may take up lots of time to calibrate all the gas detecting devices on-site.

The present invention in relation to a method and system for calibrating gas detecting device allows users to calibrate multiple gas detecting devices at a time. This may reduce the amount of time required to calibrate all the gas detecting devices on-site. Furthermore, the system may also carry the ability to alert responsible authorities remotely and automatically of the defunct devices. Thus, it may eliminate the need to perform status check on each device.

Examples of gas detecting device may include a metal oxide semiconductor (MOS) sensor. A MOS sensor may consist of metal oxide semiconductor such as tin dioxide on sintered alumina ceramic located inside a flame arrestor. The sensitivity to specific gases may be altered by changing the temperature of the sensing element.

Alternatively or optionally, the gas detecting device may include an electrochemical sensor which may comprise a chemical-electrical interface (such as a sensing electrode, an electrolyte, a counter electrode and a gas-permable membrane) that produces a differential voltage upon a chemical reaction/interaction between the detected gas and the interface. The differential voltage may be reflected as a change of electrical parameter of an electronic/electrical device such as a current passing through a transistor.

In yet another example embodiment, the gas detecting device may include photoacoustic sensor which may operate based on detecting an amount of the light of a particular wavelength within a spectrum being absorbed by a gas. As the photoacoustic sensing mechanism may require more sophisticated light sources/detectors and a spectrum analyzer to provide accurate detection results, MOS sensors and electrochemical sensors may be more preferable in some consumer-grade applications.

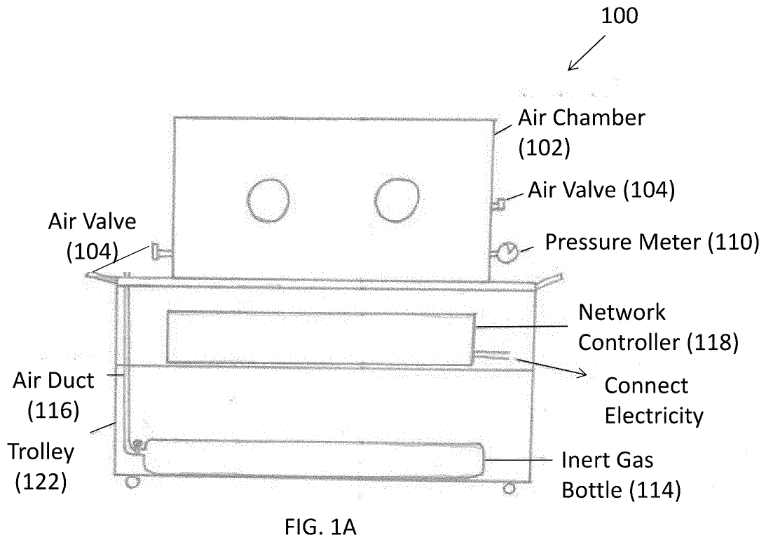

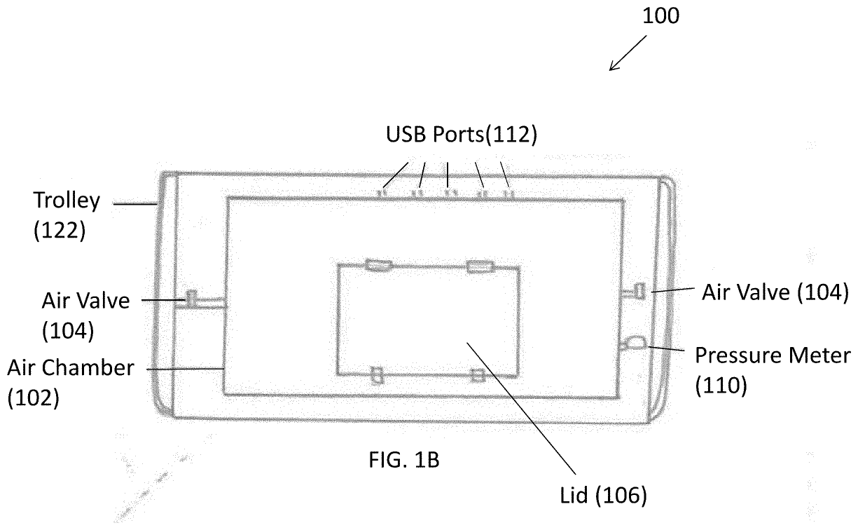

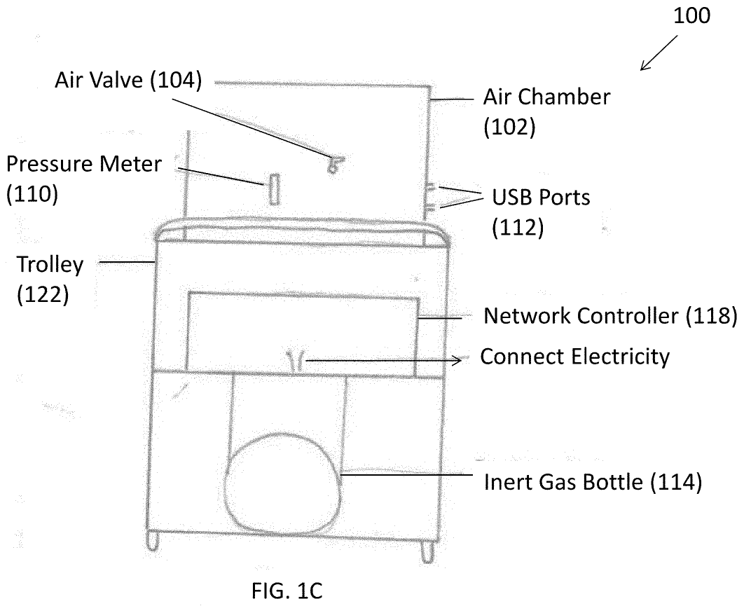

With reference to FIGS. 1a to 1c, there is provided an example embodiment of a system 100 for calibrating a gas detecting device, comprising an enclosed gas chamber 102 arranged to accommodate the gas detecting device therein, wherein the enclosed gas chamber 102 is arranged to be filled with a gas having a predetermined composition; wherein the gas detecting device is arranged to operate in a calibration mode.

In this embodiment, a box-shaped container is provided as the gas chamber 102. The box 102 may be made up of but not limited to acrylic, glass, or other composites which is preferably transparent so that visual checking can be easily done. Furthermore, the choice of material for the container should be some durable material which can withstand repeat manual operation as well as internal gas pressure. The material may also be chemically inactive, with the inner walls coated with tested catalyst such that contamination to the contained air by any sources, such as the shell of gas detecting devices, can be minimised.

The system 100 may be suitable for calibrating a gas detecting device comprising gas sensor of any type, such as but not limited to a MOS sensor and/or an electrochemical sensor.

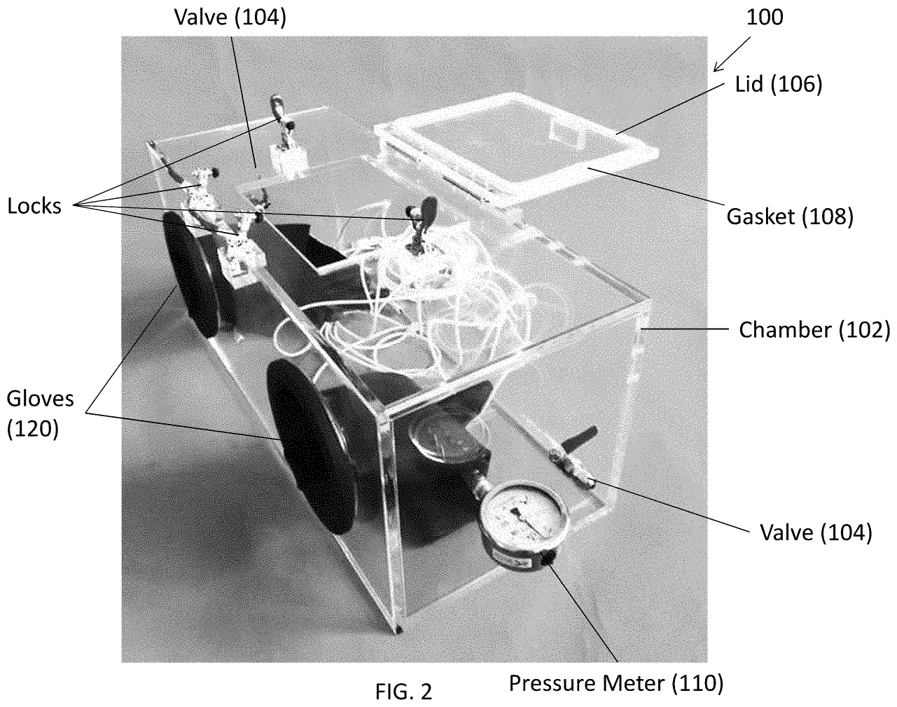

Preferably, the box 102 is of indefinite size, with respect to the number of gas detecting devices which it intends to fit. It may be larger than the box referred in FIG. 2 to fit more detectors or smaller to fit in a compact area. It may also not be cuboid shaped but in other shape with respect to the shape and required numbers of the gas detecting devices.

In an alternative embodiment, the gas chamber 102 may be made out of some elastic material (e.g. plastic, other polymer etc.). Preferably, the material should be capable to house all the necessary components (e.g. gas detecting devices and other electronics) within and allows users to manually calibrate gas detecting devices 124. Advantageously, if the container is made of elastic material, it may be light weight and easily folded as compared to cuboid containers for example. Therefore, the advantage of using elastic material is that it can be carried or transported around easily comparing to other solid containers.

The gas chamber 102 consists of a gas flow regulator connected to the box, or in the embodiment with reference to FIG. 2, at least two regulators such as valves 104, which can be used as an inlet for injecting air/gas into the chamber or as an outlet to deplete air while maintaining the desired flow rate and amount. Each valve 104 may be provided with a tap which can be actuated or by other means activated mechanically to open the channel, allowing the interior of the chamber 102 to be in fluid communication with the surroundings, open air or controlled gas environment connected with it. The valves 104 may be located at each opposite end of the box for uniform and thorough gas diffusion in the chamber 102.

The goal of the valve 104, over and above controlling the inflow and outflow of gas, may make the process as efficient as possible. The selection of valve 104 (e.g. air/vacuum valve, air release valve, combination valve etc.) may be based on factors such as whether the valve 104 is to release large and/or small amount of gas. This in turn may be based on the size of the gas chamber 102. Since the size of gas chamber 102 may vary owing to different needs, the type of valve 104 to be employed may also vary accordingly.

Referring to FIGS. 1b and 2, the chamber 102 comprises a lid 106 or a door on the top plane of the box 102. The lid 106 can be opened and closed about a hinge, and be locked tightly by the at least one locking mechanism. The lid 106 may also be layered with a rubber gasket 108, an o-ring, or any other material along the edge so to completely seal off the box 102 upon closing and locking such that the gas chamber may be fully enclosed. The lid 106 provides an entrance into and an exit from the chamber 102 for the gas detecting devices.

In an alternative embodiment, the entire chamber may be some set up similar to a lunch box with a separate lid rather than a lid attached to the box by a hinge. Similar to the construction demonstrated in FIGS. 1b and 2, the lid may also be layered with some material (e.g. o-ring, plastic gasket etc.) that can completely isolate the internal environment from the atmosphere. The lid is fastened atop the box by mechanism just as clip. Advantageously, with this setup, the opening may be bigger in comparison, thus it may allow bigger gas detecting devices to pass through the opening.

In cases where the container is made out of some elastic material as aforementioned, a different mechanism which provides an entrance into and an exit from the chamber 102 may be employed. Preferably, such mechanism should allow air tight seal and passageway for various objects passing through the opening. For example, a plastic zipper system may help seal the elastic container, thus creating an air tight space within the container.

In addition, a measuring device, such as a pressure meter 110 or a flow meter may also be installed to the chamber 102 and by a valve 104 respectively. The measuring device may inform the user of the amount and/or pressure of air within the chamber box 102, thus prompting a response from the user to regulate the valve 104, such that the pressure within the box 102 is a bit higher than the surrounding but not in excessive as required by some example applications.

Regulation of gas flow may also be achieved by other methods over and above the use of gas valve 104. Rather than implementing the regulator with mechanical means, some electrical devices may also achieve the same objective. Preferably, it should be capable to keep track of the influx or outflow of gas and is able to regulate the amount of gas passing through. To achieve this, for example, the regulation system may be constructed with pressure sensors gauging the pressure at the inlet and outlet of the gas chamber 102. Should there be excessive or insufficient gas passing in or out, the sensor may dilate or contract the tube or other means to regulate the influx or outflow of gas.

By regulating inflow and outflow or gas by electrical means, advantageously, the regulation can be done automatically with the use of some feedback mechanism. Thus, it may help maintain a more accurate and precise pressure within the gas chamber 102.

In cases where the container is made out of some elastic materials as aforementioned, users may opt not to have the pressure measuring device installed. Since the container is elastic, the manufacturer may opt to choose some material which can only encapsulate certain amount of gas within. Once the internal gas pressure reaches the maximum capacity that the elasticity of the material allows, no more gas would be allowed to enter into the chamber 102. Therefore, the gas pressure can easily be monitored and held constant by having the container holding full gas capacity.

With reference to FIGS. 1b and 1c, optionally or additionally, one or more USB charging ports 112 may be installed on a wall of the box 102. The USB charging ports 112 may be used to power or recharge the gas detecting devices within the enclosed gas chamber 102. For example, these USB ports 112 may be connected to a power source which may be an individual electricity source (such as a power bank) collectively combined within the calibration system 100 or an external power outlet wherein the power is directed by an electrical cable to the charging ports 112.

Preferably, the USB ports or any other suitable connection ports may also facilitate signal communication between the gas detecting device enclosed within the air chamber 102 and external devices, such as a controller, a computer or a handheld computing device, through a wired connection.

In another embodiment, the device may be charged by other methods such as wireless charging. The inductor may then transfer the electric power generated by induction to a power bank which may be directly connected to it.

In an alternative embodiment, the gas chamber 102 may carry a solar panel for providing additional power for the any or all the electrical components within. The solar panel may be mounted on any side of the chamber 102. This is advantageous as users normally calibrate the devices in an environment with plenty of light, the solar panel may serve as an auxiliary power for the electrical devices in most of the time.

With reference to FIG. 1A, an inert gas cylinder 114 may be placed at the bottom of the whole system 100. The inert gas cylinder 114 may be made out of material which can withstand the internal gas pressure (e.g. plastic, iron etc.). Such a cylinder 114 may securely contain a type of inert gas of industrial or analytical grade purity and with predetermined composition like but not limited to nitrogen, argon and carbon dioxide.

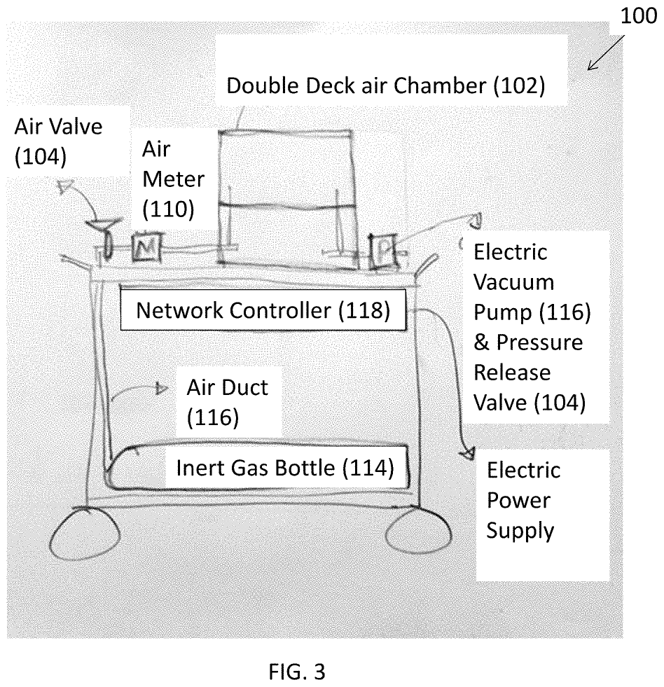

The inert gas may be directed to the chamber 102 from the cylinder 114 via an air duct 116 made of rubber, plastic, metal or other tubing that are strong enough to hold the gas with certain pressure. The inert gas may enter the chamber 102 through the inlet valve 104, fill up the chamber 102 and leave through the outlet valve 104 by the built-up pressure or in some embodiments, by the suction force generated by an electric vacuum pump 116 such as that in FIG. 3.

In another embodiment, a container, apart from cylinder 114, of different shapes or materials may be utilized as long as it can contain a specific amount of gas and withstand the pressure gas stored within. The aforementioned gas container may be placed anywhere within the system 100, or anywhere external to the system 100. If the container is built as a component of the air chamber 102, an air duct 116 of much shorter length may be utilized. Alike the previous embodiment, some mechanism controlling the inflow and outflow of gas into and out of the chamber 102 may be employed. Such mechanism does not limit to valve as long as it has the ability to control the flow of gas.

A network controller 118 may be placed at a close proximity to the chamber 102, such as in a drawer underneath the chamber 102. The network controller 118 may connect to the network-enabled gas detecting devices by wires or wirelessly, such as but not limited to Bluetooth and Wi-Fi. Multiple networkable gas detectors in the chamber 102 can simultaneously be connected to and controlled by the controller 118, such that a calibration mode can be activated remotely on individual detectors and all data received from the detecting devices while calibrating can be centralised and analysed, so to rely less on manual operation and observation.

In another embodiment, the network controller 118 may be integrated into one of the network-enabled gas detecting devices 124. Such device would then be the master device which can be activated remotely. Alike the stand-alone network controller 118 as mentioned, such master device may connect to other gas detectors by wires or wirelessly.

In another embodiment, the network controller 118 may be software installed in all the gas detectors and on computers. In this regard, a stand-alone network controller 118 may be made redundant.

Alternatively, an infrared (IR) remote controller may be used to control each of the gas detecting device placed within the transparent gas chamber using IR sensors in each of the gas detecting devices. Any other suitable wireless technologies such as RF and NFC may also be used to trigger the gas detecting devices to enter the calibration mode.

Yet alternatively, a wireless-controllable hub may be placed together with the gas detecting devices in the gas chamber, and each of the gas detecting devices may be connected to the hub with an individual wired or wireless connection. Instead of controlling each of the gas detecting device, the network controller 118 or an external controlling device may only communicate with the hub placed in the gas chamber, and the hub may then trigger the gas detecting devices to enter the calibration mode in response to the controlling signal received from the external controlling device or the network controller 118.

In an alternative embodiment, the gas detecting devices 124 may carry a function (e.g. bluetooth) which enables them to automatically pair and communicate with the chamber 102. Advantageously with this function, once they are paired out, the gas detecting devices 124 can be automatically switched to calibration mode without having the need of manual switching between calibration and regular mode.

With reference to FIG. 2, for purposes of activating and recording non-networkable gas detecting devices, the airtight chamber 102 may be designed as a glovebox. Users can activate and change the settings of the contained gas detecting devices manually using the gloves 120, all without drastically changing the gas pressure within.

Yet in other embodiments, as shown in FIG. 3, gloves 120 can be made out of any material suitable for calibrating gas detecting devices in an environment of inert gas. The gloves 120 can be excluded and double decks or more can be introduced to maximise the space in which gas detecting devices can be placed, given that all devices are networkable.

Should the gloves be removed from the gas chamber, various mechanisms may be employed to forestall gas leakage through the hole. For example, the mechanism can be a lid attached to the gas chamber with hinge. The lid can be opened and closed about a hinge, and be locked tightly by the at least one locking mechanism. The lid may also be layered with a rubber gasket, an o-ring, or any other material along the edge so to completely seal off the box upon closing and locking such that the gas chamber may be fully enclosed.

The decks may be patterned with pores so to allow fluid communication between all layers. This may increase the cost efficiency and working capacity of this calibration system.

All the components of the calibration system 100 mentioned above, including the gas chamber 102, gas cylinder 114 and the network controller 118 may be carried and transported collectively on a trolley 122 or on a mobile mechanical structure. With reference to FIG. 1a, the chamber 102 may be placed at the top of the trolley 122 with a height at around a person's chest. Underneath the chamber 102 may be a drawer which accommodates the network controller 118, while the gas cylinder 114 may be placed at the bottom of the trolley 122. This increases the mobility of the calibration system 100 and facilitates easier storage. Alternatively, other mechanical structures such as a rack or a frame may be included for accommodating different components of the calibration system 100.

In an alternative embodiment, the network controller 118, inert gas bottle 114 and the air duct 116 may be strapped onto the air chamber 102 instead of being placed on a trolley. This may be achieved by strapping the aforementioned components by rope, plastic band, or even other structures built to hold all those components and the air chamber 102 together. Advantageously, such design may reduce the overall size of the system, thus facilitates easy transportation.

To load the calibration system 100 onto the trolley, users may opt to fixate it on the trolley with screws, straps or other methods that can hold it tightly. Advantageously, this may ensure safe transportation of the calibration system 100. In an alternative embodiment, the trolley may provide some mechanism such as magnet which can hold the system 100 in place without requiring users manually fastening it onto the trolley.

The calibration system 100 as mentioned above can be carried around by other means apart from trolley. Other transportation methods can be, for example, car, truck, drone etc. Any method would do as long as it facilitates easy transportation of the calibration system 100.

In another alternative embodiment, the calibration system 100 can itself be an integral part of a trolley, car, truck, drone etc. In other words, the calibration system 100 is an inseparable part of the trolley system. Advantageously, users do not have to manually load and unload the calibration system 100 onto the trolley every time before and after transportation.

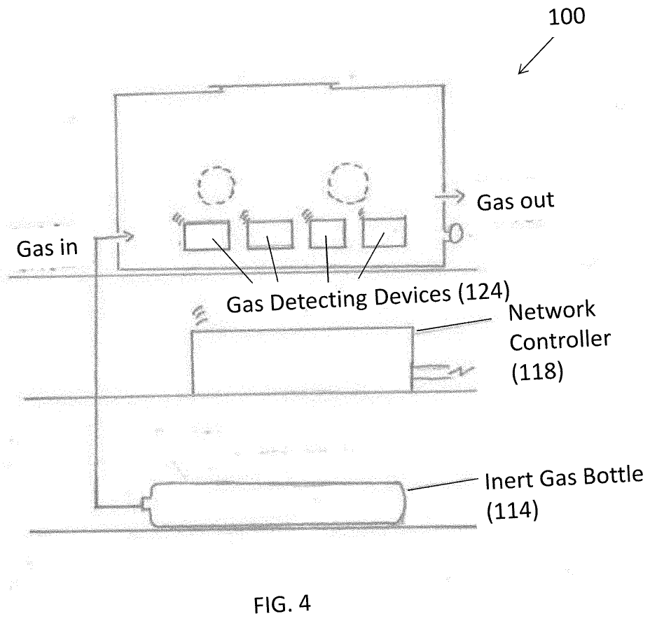

With reference to FIG. 4, there is shown an example setup during the calibration process includes placing a few gas detectors 124 into the chamber box 102, after which the inert gas enters the inlet and leaves at the outlet valve 04.

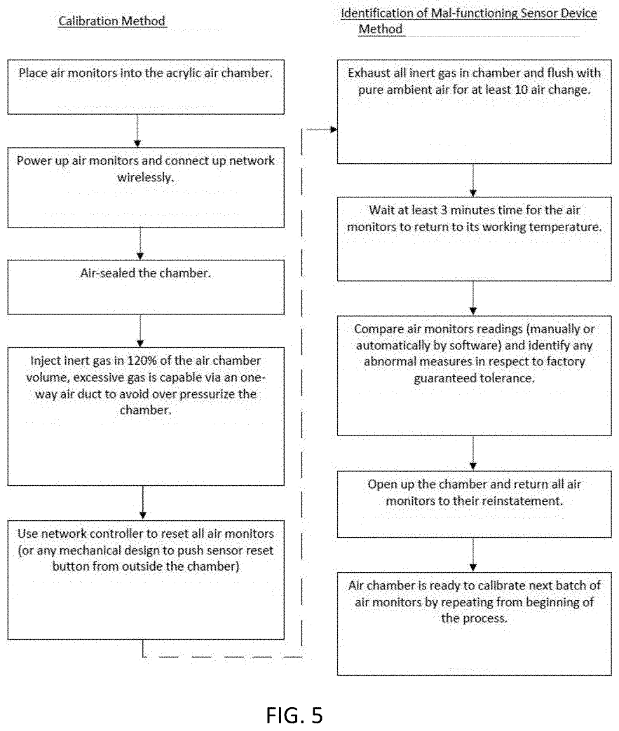

With reference to FIG. 5, in order to perform the calibration process, the gas detecting devices 124 that require calibration may be stacked and placed into the chamber 102 without blocking the gas flow to their individual measuring tip. Optionally, users can stack multiple gas detecting devices 124 in the chamber 102 up until it is fully loaded. Advantageously, the number of gas detecting devices allowed is only limited by the size of the chamber 102. However, user may choose other size of chamber 102 based on their needs.

After the gas detecting devices 124 are carefully stowed in the chamber 102, user may then seal the lid 106 with a locking mechanism provided. Should the lid 106 is properly sealed, the locking mechanism may give off an electrical signal (e.g. a flashing light or a beep sound) or a mechanical signal (e.g. clutch sound).

Inert gas with purity of industrial or analytical grade is then directed from the inert gas cylinder 114 to the inlet valve 104 via an air duct 116, so to replace the air in chamber 102 with excess inert gas. The inert gas to be used should not be equivalent to which the gas detecting devices are intended to measure, for example, nitrogen may be used for the calibration process of the gas detectors which may be designed to detect volatile organic compounds (VOC).

The system for calibrating the gas detecting devices 124 may be operated as follows: contained gas is replaced with inert gas by injecting excessive inert gas into the chamber 102 while pushing out the original air through the outlet valve 104. The outlet valve 104 is then closed off to build up the pressure within the chamber 102 up to around 120% of volume of inert gas, before closing the inlet valve 104. Yet in an alternate embodiment, a vacuum pump 116 may be used instead to first extract the air out of the chamber 102 before injecting the inert gas. Both are feasible methods of filling inert gas but the previous one is preferred as long as the inert gas supply is not a concern, so to reduce the operation cost.

The calibration can then begin by switching the gas detecting devices 124 to calibration mode, either manually with the gloves 120 provided or other mechanical means, or collectively through a wired or wireless network connecting the powered network controller 118 with a centralised control platform. The zero baseline of specific gas type is redefined according to the exposed gas content.

In addition, screening of defective gas detecting devices 124 may be performed after the calibration process. Preferably, the method of identifying a malfunctioning gas detecting device, comprising the step of: calibrating a gas detecting device; replacing the inert gas in the gas chamber with standard gas including standard gas composition; and determining whether the gas detecting device is malfunctioning based on a comparison of a detection result associated with the standard gas composition and a predetermined record.

Preferably, by filling a gas such as a standard gas including a standard or a known gas composition, the reading on the calibrated gas detecting devices should match with the known gas composition and preferably within a factory guaranteed tolerance.

After opening and exhausting the contained inert gas through the outlet valve 104, the chamber 102 is washed with atmospheric air repeatedly, e.g. for at least ten times, to restore normal gas content. Upon the stabilisation of temperature and pressure after at least three minutes, the intensity readings given by each gas detecting device 124 are recorded and compared manually or through the centralised network. Devices giving reading of substantial deviation from peers or factory settings are considered faulty thus should be retired.

Should a gas detecting device 124 be found defective, there can be various ways to notify relevant authorities. This can be achieved by giving off an electrical signal (e.g. a flashing light or a beep sound) or a mechanical signal (e.g. clutch sound). Over and above this, defective gas detecting devices 124 may send a signal with its identity (e.g. specific pin number) to the network controller 118. The network controller 118 may then gather all the identities of defective gas detecting device 124 and send the data wirelessly (e.g. via Wi-fi) to relevant authorities to prompt further response.

Advantageously, since persons who calibrate the gas detecting devices 124 may not be responsible for replacing the defective devices 124, this function enables immediate action once a defective gas detecting device is found 124. Thus, it may eliminate errors in manually recording the identities of defective devices 124.

As for the functioning devices 124, they can be reinstated and the calibration system is ready for the next batch.

These embodiments may be advantageous in that several gas detecting devices, as in hundreds can be simultaneously calibrated with a reduced operating cost, due to lower gas consumption per batch when compared to the total gas use by the gas kits offered in the market, which are designed for one-to-one calibration. The equipment is commercially available, and the gas cylinder can be easily and safely acquired and transported such that no special license is required for operation.

Advantageously, due to a controlled gas environment, this calibration system also offers greater accuracy and quality than open-air calibration, and that a network controller can handle a great number of calibration works at limited time and man power.

Design of the invention can easily be adjusted and customised as shown in the different embodiments to meet the needs of calibration amount and user's requirements, such as the size, shape and extra features of the gas chamber. The whole system can also be implemented onto a trolley for higher mobility and easier storage, and offers an alternative on-site calibration method due to a huge tolerance of a sealed off chamber to the open air quality, temperature, pressure, and humidity.

In addition, this invention allows peer comparison thus identification of defective devices which is not provided from other current calibration methods. Furthermore, if the gas detecting devices carry remote communication capability and are communicable with each other and a master device or a server, the result of identification of defective device could be immediately reported to any authority. In other words, the authority can remotely monitor the status of all the gas detectors rather than checking one by one on-site. This can simplify the process of identifying defective devices, thus it may potentially reduce the maintenance cost.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A method of calibrating a gas detecting device, comprising the steps of:
providing the gas detecting device in an enclosed gas chamber filled with a gas having a predetermined composition; and
activating the gas detecting device to operate in a calibration mode.

2. The method of calibrating a gas detecting device in accordance with claim 1, wherein the enclosed gas chamber is filled with an inert gas.

3. The method of calibrating a gas detecting device in accordance with claim 1, further comprising the step of injecting the gas to fill up the enclosed gas chamber and depleting air in the enclosed gas chamber, wherein a gas pressure within the enclosed gas chamber is higher than that of the environment.

4. The method of calibrating a gas detecting device in accordance with claim 3, further comprising the step of adjusting the gas pressure within the enclosed gas chamber.

5. The method of calibrating a gas detecting device in accordance with claim 1, wherein the step of activating the gas detecting device to the calibration mode further comprising the step of wirelessly controlling the gas detecting device with a wireless controller.

6. The method of calibrating a gas detecting device in accordance with claim 1, wherein the step of activating the gas detecting device to the calibration mode further comprising the step of manually manipulating of the gas detecting device enclosed within the gas chamber.

7. The method of calibrating a gas detecting device in accordance with claim 1, further comprising the step of providing at least one additional gas detecting device in the enclosed gas chamber such that the gas detecting device and the at least one additional gas detecting device are simultaneously calibrated.

8. The method of calibrating a gas detecting device in accordance with claim 1, further comprising the step of transporting a mobile calibration system including the gas chamber and a gas cylinder which contains the gas.

9. The method of calibrating a gas detecting device in accordance with claim 8, wherein the gas chamber and the gas cylinder is carried on a mobile mechanical structure.

10. A method of identifying a malfunctioning gas detecting device, comprising the step of:
calibrating a gas detecting device using a method in accordance with claim 2;
replacing the inert gas in the gas chamber with standard gas including standard gas composition; and
determining whether the gas detecting device is malfunctioning based on a comparison of a detection result associated with the standard gas composition and a predetermined record.

11. The method of identifying a malfunctioning gas detecting device in accordance with claim 10, wherein the predetermined record includes a factory guaranteed tolerance.

12. The method of identifying a malfunctioning gas detecting device in accordance with claim 10, further comprising the step of waiting for a predetermined period of time prior to recording the detection result.

13. The method of identifying a malfunctioning gas detecting device in accordance with claim 10, wherein the step of replacing the inert gas in the gas chamber with standard gas comprising the step of flushing the gas chamber with the standard gas for multiple times.

14. A system for calibrating a gas detecting device, comprising an enclosed gas chamber arranged to accommodate the gas detecting device therein, wherein the enclosed gas chamber is arranged to be filled with a gas having a predetermined composition; and wherein the gas detecting device is arranged to operate in a calibration mode.

15. The system for calibrating a gas detecting device in accordance with claim 14, wherein the enclosed gas chamber is filled with an inert gas.

16. The system for calibrating a gas detecting device in accordance with claim 14, further comprising a gas supply arranged to inject the gas to fill up the enclosed gas chamber and to deplete air in the enclosed gas chamber, wherein a gas pressure within the enclosed gas chamber is higher than that of the environment.

17. The system for calibrating a gas detecting device in accordance with claim 16, wherein the gas supply includes a gas cylinder.

18. The system for calibrating a gas detecting device in accordance with claim 16, further comprising at least one valve and/or regulator arrange to control a fluid communication between the gas chamber and the gas supply.

19. The system for calibrating a gas detecting device in accordance with claim 14, further comprising a wireless controller arranged to wirelessly control the gas detecting device.

20. The system for calibrating a gas detecting device in accordance with claim 14, further comprising a flexible structure provided on a wall of the gas chamber arranged to facilitate a manual manipulation of the gas detecting device enclosed within the gas chamber by a user.

21. The system for calibrating a gas detecting device in accordance with claim 20, wherein the flexible structure includes a glove.

22. The system for calibrating a gas detecting device in accordance with claim 14, wherein the enclosed gas chamber is further arranged to accommodate at least one additional gas detecting device, wherein the gas detecting device and the at least one additional gas detecting device are simultaneously calibrated.

23. The system for calibrating a gas detecting device in accordance with claim 14, further comprising a mobile mechanical structure arranged to accommodate the gas chamber, the wireless controller and the gas supply.

24. The system for calibrating a gas detecting device in accordance with claim 14, further comprising a standard gas supply arranged to facilitate a determination of a malfunctioning gas detecting device.

25. The system for calibrating a gas detecting device in accordance with claim 14, wherein the gas detecting device comprises a metal oxide semiconductor sensor and/or a electrochemical sensor.

26. A system for calibrating a plurality of gas detecting devices, comprising:
a gas chamber arranged to accommodate the plurality of gas detecting devices, wherein the gas chamber includes a plurality of valves arranged to facilitate a fluid communication between an internal cavity of the gas chamber and an external environment or a supply of an inert gas, and a flexible structure provided on a wall of the gas chamber for facilitating a manual manipulation of the plurality of gas detecting devices enclosed within the gas chamber by a user;
a wireless controller arranged to wirelessly control the plurality of gas detecting devices disposed within the gas chamber; and
a mechanical structure arrange to accommodate the gas chamber, the wireless controller and a container arranged to supply the inert gas to the gas chamber;
wherein the gas chamber is arranged to define an enclosed gas chamber filled with the inert gas having a predetermined composition so as to facilitate a calibration process performed by each of the plurality of gas detecting devices operating in a calibration mode in response to the control by the wireless controller or the manual manipulation by the user.
